(19) United States
(12) Patent Application Publication
(10) Pub. No.: US 2004/0226562 A1
(43) Pub. Date: Nov. 18, 2004

(54) Blower assembly for CPAP

(76) Inventor: Steven S. Bordewick, North Shoreview, MN (US)

Correspondence Address: Kathleen R. Terry, 2417 Como Avenue, St. Paul, MN, 55108-1459, US

(21) Appl. No.: 10/729,359

(22) Filed: Dec. 4, 2003

Related U.S. Application Data
(60) Provisional application No. 60/431,577, filed on Dec. 6, 2002.

(51) Int. Cl. A61M 16/00; A62B 7/00

(52) U.S. Cl. 128/204.23; 128/204.18; 128/204.21

(57) ABSTRACT

This invention relates generally to a blower assembly for providing continuous positive airway pressure (CPAP) to a patient. A centrifugal blower is contained inside a compartment within a container which is designed to operate with minimal noise and vibration. A separately contained muffler system at the gas inlet to the blower dissipates the turbulence of the incoming gas to convert the flow characteristics to approximately laminar flow. A muffler system may also be provided at the gas outlet. The pressurized gas may be ambient air, oxygen or a mixture thereof. The power source may be direct or alternating current. Controls are provided for adjusting air flow. The air pathway within the system may be reduced in cross sectional area

### RELATED APPLICATIONS

This application takes priority from Provisional Application Ser. No. 60/431,577, filed Dec. 6, 2002.

### FIELD OF THE INVENTION

This invention relates generally to a blower assembly for providing continuous positive airway pressure (CPAP) to a patient.

### BACKGROUND OF THE INVENTION

As a person ages, airway patency may be reduced due to loss of muscle tone in the muscles of the throat which normally serve to prevent the tissues from impinging on the airway. This condition is more severe in patients who are obese, have naturally narrow airways or airways that may be partially blocked by tonsils, soft palate or uvula. The result can be snoring as the tissues vibrate as air is forced through the narrowed airway under heightened pressure. Snoring is disruptive to sleep; the snorer not only keeps companions awake but will himself awaken many times in the night. This sleep disturbance leads to a feeling of tiredness or exhaustion during the day and a decrease in mental and physical performance.

In about a third of snorers, the condition can be lethal. When narrowing of the airway proceeds to complete occlusion, the condition is termed sleep apnea. A faulty feedback loop between the brain and the respiratory system lets the airway completely collapse until the brain registers low oxygen levels and the patient jerks awake to resume breathing. These constant jump starts, which can happen as often as twice a minute, send the heart rate on a roller coaster. In susceptible patients, high heart rates can bring on fatal arrhythmia or myocardial infarction.

Continuous positive airway pressure (CPAP) is a method in use for some time to alleviate the symptoms of snoring or sleep apnea by delivering air or gas under a pressure sufficient to create a pneumatic splint and thereby mimic the effect of the natural waking tone of the throat muscles in holding the soft tissues around the airway from partially or totally occluding the airway passage. Patients other than those with airway problems can benefit from CPAP. These groups include patients with weakened respiratory muscles who cannot pull air into their lungs, such as those with post polio or amyotrophic lateral sclerosis disease, patients with traumatic nerve damage or adult respiratory distress. CPAP apparati are generally comprised of a blower for providing a stream of air or gas under pressure, a mask and tubing to connect the mask to the blower source.

A blower assembly suitable for CPAP consists of a fan, sensors and valves to control pressure and flow and tube connections to a face mask. A preferred fan is a centrifugal fan, that is, one in which the stream of gas enters and then exits the fan at right angles. Air flow resistance in the airway varies as a patient breathes, being lower during expiration and higher during inspiration. Therefore, it has been found that varying the pressure applied, most easily by varying the rotation of the blower fan, is more comfortable to a patient. Another way of varying the pressure is by a series of valves. In some models, the fan may run continuously at a constant rotation while a valve closes partially or completely when the sensor registers a reduced resistance to air flow, that is, when the patient exhales, and opens when a higher pressure is required to maintain airway patency. This so-called bi-level triggering is said to be more tolerable to the patient as it mimics natural changes in airway resistance. Triggering has also been achieved by use of a microphone to “hear” inhalation and exhalation. In the same manner, some devices may be set to “ramp up” over the first 20 minutes or so of use, avoiding the feeling of pressure while the patient is awake. Others are equipped with computer programmed controls for autotitrating the minimal pressure at an instant time that is necessary for airway patency.

Whatever the model blower unit, operation tends to be noisy due to the turbulent airflow (aerodynamic noise) and the moving blades of the blower (mechanical noise). The blower will also cause the unit to vibrate. Both noise and vibration are abated by such means as rubber feet, insulation and extending the airflow pathway. For example, one device in common employs sheets of foam rubber to line the blower box and along the spiral air pathway. For quiet operation, these sheets are generally approximately at least ten millimeters thick and are applied on all six interior surfaces, thereby increasing the volume of the unit. All of these means take up space, resulting in a large blower unit. Such a unit is too large and heavy to be readily portable and further, takes up considerable space on the patient's bedside table.

The need remains for a small, self-contained blower unit that is quiet in operation, convertible to battery power and portable.

### SUMMARY OF THE INVENTION

The present invention provides a quiet, self-contained blower assembly for CPAP that is provided with a noise reduction system in an inlet muffler box, which is isolated from the blower, an outlet box, which is isolated from the blower, and a control compartment, thus separating aerodynamic and mechanical noise.

In one embodiment of the invention, the noise reduction system is comprised of an isolated inlet muffler box, wherein the intake gas goes into a first perforated tube, which serves as a baffle, sealed at the end distal to the inlet orifice. The incoming air is directed through the perforations and passes through the compartment and through the perforations of a second perforated tube with a sealed end and thence out an orifice into the blower. A divider is placed along the length of each perforated tube to direct the air flow so that it makes right angle bends before entering the perforations of the second perforated tube, which is likewise provided with a divider along its length. In a preferred embodiment of the invention, the air pathway is lengthened by placing a divider between the inlet perforated tube and the outlet perforated tube. The perforated tubes and the dividers form a baffle system which directs the airflow along this lengthened air pathway of this inlet muffler box. The inlet and outlet orifices are preferably placed on the same wall of the muffler system but may be placed on any wall as long as dividers to form a baffle system are provided to lengthen the air pathway. The baffle is preferably comprised of a rectangular divider placed midway between the inlet and the outlet orifices and occluding approximately two-thirds of the air pathway, thus establishing a further lengthened airway passage. The muffler box is preferably lined with an anechoic material.

The perforated tube may be round, square or rectangular in cross section and has at least ten perforations of about four millimeters in diameter. The preferred tube material is elastomeric polymer, rubber, foam or fiber mesh material. When fiber mesh is used, the spaces within the mesh may serve as perforations.

The outlet system of the invention may be a compartment lined with anechoic material where the airflow enters, makes a right angle turn and exits to the hose leading to the face mask. The outlet box may also be provided with a muffler system such as that of the inlet box.

The anechoic material is any material that is sufficiently porous or textured on the surface to provide void spaces that reflect internally and finally absorb sound waves. Anechoic materials include foam sheeting, textured and acoustic paints.

The most preferred embodiment comprises an air pathway within the blower that is reduced in cross sectional area from the cross sectional area of the hose leading to the patient. This embodiment has a connector at the outlet orifice of the outlet box to mate the smaller orifice with the larger hose. The air pathway may be 20% to 50% reduced in cross sectional area from the cross sectional area of the hose leading to the patient. More preferably, the cross sectional area is reduced 25% to 35%. Most preferably, the cross sectional area is reduced 30%.

The control compartment comprises a connection to a power supply, a primary and secondary voltage controller, a blower fan and a blower control. The power supply may be either alternating line current or direct battery current. The primary and secondary voltage controllers adjust the appropriate voltage for each component, namely the fans and the sensors. The blower controller adjusts the speed of the blower fan in order to increase or decrease the air pressure. A system controller is provided to set the speed of the blower fan. The system controller sends signals to the display panel so as to show values.

In one embodiment of the invention, the power supply is an alternating current through line voltage. In another embodiment of the invention, the power supply is a battery, preferably a rechargeable battery. In one embodiment, the battery is permanently and operably attached to the blower assembly. In another embodiment, the battery is detachable from the blower assembly.

Any embodiment of the blower assembly may be fitted with a humidifier unit. The unit comprises a container of water over which the stream of air passes. The humidifier is preferably placed near the outlet orifice and may be situated on the outside of the container just at the orifice or at any point along the hose up to its attachment to the face mask. The container for water is preferably detachable for convenient refill and cleaning.

The blower assembly may be provided with a carrying case for portability.

### DESCRIPTION OF THE DRAWINGS

FIG. 1 is a block diagram of the blower assembly (CPAP).

FIG. 2A is a diagram of the inlet muffler box. FIG. 2B is a diagram of the outlet muffler box.

### DETAILED DESCRIPTION OF THE INVENTION

For purposes of describing this invention, the term “airway” refers to the patient's pulmonary system while “air pathway” is used to describe the air flow within the CPAP and leading to the patient. The term “air” includes ambient air and gas, such as liquid or cylinder oxygen.

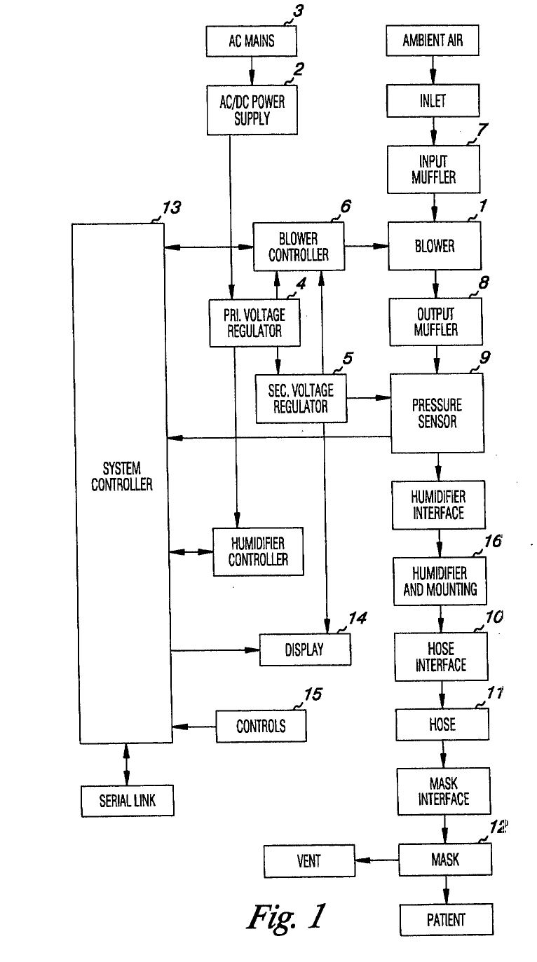

FIG. 1 shows a functional block diagram with the components for a blower assembly suitable for CPAP. The blower 1 is powered by an electric current supplied by an AC/DC power supply 2. When the power supply is AC, a main plug 3 leads to line current. The power supply is in line with a primary voltage regulator 4 and a secondary voltage regulator 5. The rotation of the blower fan 1 is controlled by an electronic circuit 6 forming a blower controller. In operation, the blower 1 receives gas from the inlet muffler box 7. The pressure established at the output muffler 8 orifice is sensed by a pressure transducer 9. The stream of pressurized gas is then directed through a connection 10 to a hose 11 leading to a face mask 12.

The sensors feed into a system controller 13 with a display 14 and controls 15 for adjusting fan speed and thereby gas pressure. Controls 15 may be changed manually or by software or by both means.

The blower assembly will be supplied to a patient fully assembled and preset to an average outlet pressure. In use, a patient dons a face mask, most preferably supported by the mask support described in co-pending U.S. Provisional Patent Application 60/404,685, attaches the hose from the blower assembly to the mask and goes to sleep. The preset outlet pressure is initially at a low value, from about four to six cm water and increases over time to about 10 to 20 cm water. The time of increase may be from ten minutes to half an hour or so, depending on the patient's preference and the time required to fall asleep. The increase may be set to be stepwise, continually variable or hyperbolically variable. The actual values will vary and be adjusted to be the minimum necessary to keep the airways patent for each CPAP user. In one embodiment of the invention, once the maximum pressure is reached, the pressure is held constant by varying the fan speed.

In a bi-level embodiment of the invention, the pressure sensor 9 feeds back to the blower controller 6 and when it senses that the pressure in the system, the airway resistance, drops, that is, the patient exhales, it reduces the fan speed according to a preprogrammed amount and returns to the preset pressure when the airway resistance increases on inspiration. This bi-level embodiment is adequate for the usual patient. If the patient experiences sporadic complete obstruction at the preset pressure, the pressure sensor 9 senses increased pressure in the system and the blower controller 6 increases the fan speed by whatever amount is necessary to overcome the increased resistance. This embodiment may be termed the continuously variable self-sensing CPAP. The system controller 13 makes the necessary adjustments and displays the values on display 14. Controls 14 may be used to manually or by program direct the system controller.

Regarding FIG. 1, a preferred CPAP has a humidifier 16, preferably placed past the output muffler box and before the hose interface 10. The humidifier may be placed at any point outside the assembly or along the hose, where it is easily accessible for filling and cleaning.

In the preferred placements of the components, the inlet muffler box is placed next to the blower box and the outlet muffler box is placed on the opposite side of the blower box. A connector nozzle integrates the reduced cross-sectional air pathway within the CPAP with the larger cross sectional area of the hose. The inlet muffler box is generally lower in height than the blower box and leaves space for the control box to rest on top of it. The depicted placement and dimensions of the boxes result in a compact rectangle, although any placement and dimensions comprising the components of the invention are considered to be insubstantial variations and are thus within the spirit and scope of this invention.

CPAP apparati have been in use for some time. When a patient is confined to bed or his home, the models presently available serve their function well. Although large and heavy, this is not a problem when used only in one location. However, many patients suffering from sleep apnea or snoring would benefit when having small, light, self-contained apparatus that can be carried easily from place to place and used where there is no power supply with line current compatible with that of the home country. When the size of the apparatus is reduced, noise and vibration become a problem. A smaller fan must be run at a higher (noisier) speed in order to attain the needed air pressure and the reduced size of the assembly cannot contain the thick foam padding used in larger models. An air pathway connecting to the hose to the patient also takes up space. The inventor has solved the problem of size reduction and noise reduction by reducing the cross sectional area of the air pathway within the blower assembly, isolating aerodynamic and mechanical noise, and further reducing aerodynamic noise by coating the interior surfaces with anechoic materials.

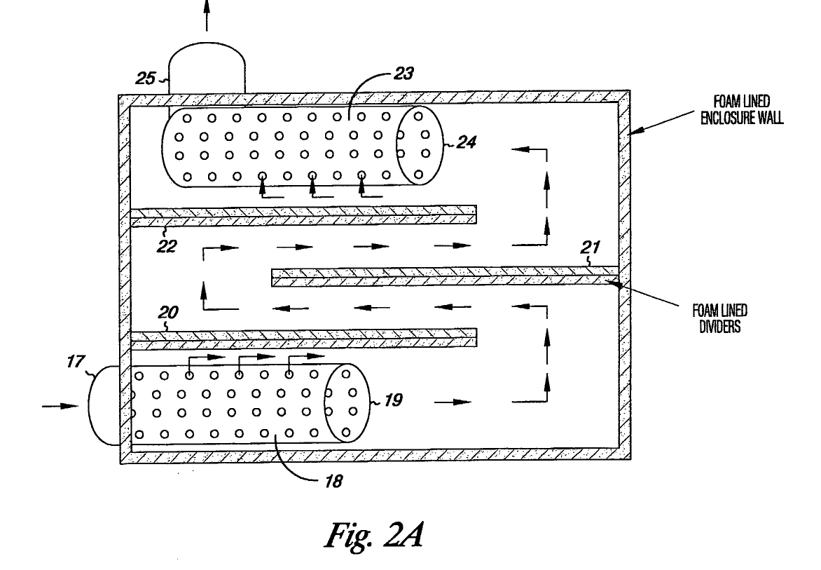

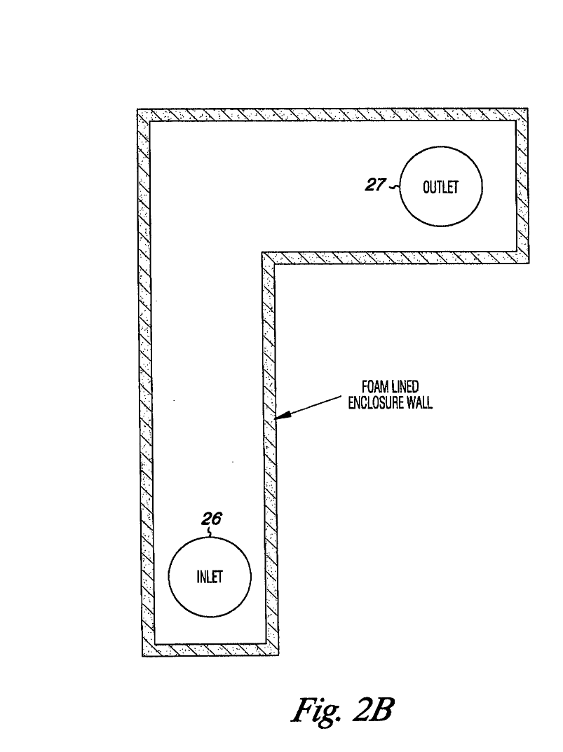

Going to FIG. 2A, the inlet muffler box receives air or gas at inlet 17. The turbulence of the air is attenuated as it passes into the first perforated tube 18 with sealed end 19. The air is directed out through the perforations and is constrained by first divider 20 to travel around the divider, make a right turn, impinging against second divider 21 and further directed around the divider 21 making a right turn, impinging against third divider 22 and turning again to exit through the perforations of second perforated tube 23 with sealed end 24, forming the baffle system, and thence out the outlet orifice 25 of the inlet muffler box to the blower box.

FIG. 2B shows the simple outlet muffler box. Air enters at the inlet orifice 26, passes through the box, which is lined with anechoic material, and exits through the connector nozzle 27. It is understood that the simple outlet muffler box may be provided with a baffle system similar to that of the inlet muffler box, if preferred.

As the air passes through the muffler boxes, its original turbulent and noisy flow is transformed into quiet laminar flow. Sound reduction is increased by coating the walls and baffles with an anechoic material. As the sound waves enter the void spaces of this material, they rebound within the void and are absorbed. Materials include sheet foam, which is effective in very thin application, textured rubber paints, epoxy paints, paints containing fibers and acoustic paints, which form voids. In general, acoustic paints are formulated to include microspheres which are fragile and break open as the paint dries, forming the preferred spherical void space. As well as the muffler boxes, the blower box and preferably all interior surfaces of the case are coated with anechoic material.

This muffler system is adequate to reduce noise of the higher fan speed used in the preferred embodiment wherein the air pathway is reduced in cross sectional area.

It is understood that following the teachings of this invention, those skilled in the art may readily, without undue experimentation, make variations and adjustments to the apparatus described herein without departing from the spirit and scope of this invention.

I claim:

1. A blower assembly for providing continuous positive airway pressure to a patient comprising
an inlet muffler box which receives a turbulent stream of air which is directed along an air pathway and is transformed into an approximately laminar stream of air;
a blower box comprising a centrifugal fan; and
an outlet muffler box connected to a hose leading to the patient.

2. The blower assembly of claim 1 wherein the interior surfaces are coated with an anechoic material.

3. The blower assembly of claim 1 wherein the air pathway is reduced in cross sectional area from that of the hose leading to the patient.

4. The blower assembly of claim 3 wherein the cross sectional area of the air pathway is 20% to 50% smaller than that of the hose leading to the patient.

5. The blower assembly of claim 3 wherein the cross sectinal area of the air pathway is 25% to 35% smaller than that of the hose leading to the patient.

6. The inlet muffler box of claim 1 which comprises
a first perforated tube having a sealed end distal to an inlet orifice which receives a turbulent stream of air through the orifice;
a first divider placed along the length of the first perforated tube;
a second divider placed on the opposite wall from the inlet orifice;
a second perforated tube having a sealed end distal to an orifice; and
a third divider along the length of the second perforated tube, and an orifice opening to the blower box.

7. The inlet muffler box of claim 6 wherein the perforated tubes are of approximately equal length and each of the dividers is about 60% of the length of the perforated tubes.

8. A blower assembly for providing continuous positive airway pressure to a patient comprising
an inlet muffler box which receives a turbulent stream of air which is directed into a first perforated tube with a sealed end, whereby the air enters the muffler box through the perforations in the first tube, is directed along an air pathway by a first divider placed along the tube, is diverted by a second divider placed on the wall of the box opposite to the first perforated tube, is further diverted by a third divider placed along a second perforated tube with a sealed end, whereby transforming the turbulent stream of air into a laminar flowing stream of air which then passes through the perforations in the second tube into a blower box; and
a blower box comprising a centrifugal fan; and
an outlet muffler box connected to a hose leading to the patient.

9. The blower assembly of claim 1 or 8 wherein the outlet muffler box comprises the inlet muffler box of claim 6.
